(19) United States
(12) Patent Application Publication
(10) Pub. No.: US 2012/0053161 A1
(43) Pub. Date: Mar. 1, 2012

(54) TOPICAL SOLUTION FORMULATIONS CONTAINING A CORTICOSTEROID AND A CYCLODEXTRIN

(75) Inventors: Ernesto J. Castillo, Fort Worth, TX (US); Huixiang Zhang, Forth Worth, TX (US); Glenn D. Stafford, White Settlement, TX (US); Wesley Wehsin Han, Arlington, TX (US)

(73) Assignee: Novartis AG, Basel (CH)

(21) Appl. No.: 13/319,311

(22) PCT Filed: Jul. 28, 2009
(86) PCT No.: PCT/US09/51955

§ 371 Date: Nov. 7, 2011

(30) Foreign Application Priority Data
May 8, 2009 (US) 12/437895

(51) Int. Cl. A61K 31/573; A61P 11/02; A61P 27/02; A61P 27/16; A61K 31/57; A61K 31/569

(52) U.S. Cl. 514/171; 514/180; 514/179

(57) ABSTRACT

Solution formulations containing a corticosteroid, cyclodextrin, and xanthan gum are disclosed. The formulations are intended for topical application to the eye, ear, or nose.

### BACKGROUND OF THE INVENTION

This invention relates to topically administrable solution formulations containing a corticosteroid and a cyclodextrin.

Many corticosteroids are known, one of which is dexamethasone. Both solution and suspension compositions containing dexamethasone as the sole active agent are marketed. The solution compositions contain dexamethasone in the form of dexamethasone sodium phosphate. The suspension formulations contain dexamethasone in the form of dexamethasone alcohol. See Ophthalmic Drug Facts '99, Facts and Comparisons, St. Louis, Mo. (1999), p. 87. Additionally, aqueous anti-inflammatory/anti-infective combination products containing dexamethasone are currently marketed. See Ophthalmic Drug Facts '99, Facts and Comparisons, St. Louis, Mo. (1999), p. 121-122. The only such combination product identified as a solution is a neomycin sulfate/dexamethasone sodium phosphate solution product.

Solution compositions containing water-insoluble forms of dexamethasone (i.e., forms other than dexamethasone phosphate) must contain a solubilizing agent. Cyclodextrins are one type of solubilizing aid that has been used with steroids. See, for example, U.S. Pat. No. 4,383,992; U.S. Pat. No. 5,229,370; and European Patent No. 0 326 196 B1.

To be commercially viable, solution compositions must remain physically stable over extended periods of time to permit manufacture, handling, storage, shipping and a reasonable shelf-life.

### SUMMARY OF THE INVENTION

The present invention provides solution compositions of water-insoluble corticosteroids. The present compositions contain a cyclodextrin as a solubilizing agent. In addition, the compositions contain xanthan gum in an amount sufficient to enhance the physical stability of the compositions.

Among other factors, the present is based on the finding that solution compositions containing a corticosteroid, a cyclodextrin and xanthan gum possess superior physical stability compared to similar formulations that lack xanthan gum or that contain polyethylene glycol instead of xanthan gum.

### DETAILED DESCRIPTION OF THE INVENTION

Unless indicated otherwise, all ingredient amounts presented as a percentage are in weight/weight units, % (w/w).

The corticosteroid ingredient of the present invention may be any pharmaceutically acceptable corticosteroid that is not sufficiently soluble in water to provide a target corticosteroid concentration in a solution composition.

Suitable corticosteroids include, but are not limited to, dexamethasone, fluorometholone, prednisolone, loteprednol and rimexolone. A preferred corticosteroid is dexamethasone in the form of dexamethasone alcohol or dexamethasone acetate. The corticosteroid ingredient will comprise about 0.01-0.3%, preferably about 0.05-0.2%, and most preferably about 0.1%.

The cyclodextrin ingredient in the compositions of the present invention may be any pharmaceutically acceptable cyclodextrin. Many cyclodextrins are known, including, but not limited to those classified as β-cyclodextrin derivatives, y-cyclodextrin derivatives and sulfated cyclodextrin derivatives. A preferred cyclodextrin is hydroxypropyl-β-cyclodextrin. The amount of cyclodextrin ingredient included in the compositions of the present invention will depend on the concentration of corticosteroid. The amount of cyclodextrin should be enough to solubilize all of the selected corticosteroid so that the composition is administered to a patient as a solution. Generally, the amount of cyclodextrin contained in the compositions of the present invention will be about 1 to 15%, preferably about 2 to 10%, and most preferably about 4 to 7%.

In addition to the corticosteroid and cyclodextrin, the compositions of the present invention contain xanthan gum. Xanthan gum is a well-known polysaccharide that is commercially available from a variety of sources. The amount of xanthan gum contained in the compositions of the present invention will depend upon the amounts of the corticosteroid and cyclodextrin ingredients in the composition, but will generally range from about 0.1 to about 0.6%, preferably 0.1-0.4%, and most preferably, 0.2-0.3%. The compositions contain an amount of xanthan gum sufficient to enhance the physical stability of the composition relative to a similar composition lacking xanthan gum.

The compositions of the present invention have a pH from 4-8. pH can be adjusted with NaOH/HCl or other pH adjusting agents known in the art. The compositions of the present invention may contain one or more buffering agents.

The solution compositions of the present invention optionally comprise a second active ingredient. Any pharmaceutically active compound that is suitable for ophthalmic, otic or nasal administration may be used. Such active ingredients include, but are not limited to fluoroquinolone antibiotics, such as ciprofloxacin, moxifloxacin, and gatifloxacin. The fluoroquinolone can be present in any pharmaceutically acceptable form such that it is in solution in the composition that is administered to a patient. A preferred fluoroquinolone antibiotic is ciprofloxacin. A preferred form of ciprofloxacin is ciprofloxacin hydrochloride, monohydrate. If present, the fluoroquinolone ingredient will comprise about 0.1-1% of the compositions of the present invention. In the case where the fluoroquinolone is ciprofloxacin, the preferred amount of ciprofloxacin in the compositions of the present invention is 0.3%. In the case where the fluoroquinolone is moxifloxacin, the preferred amount of moxifloxacin in the compositions of the present invention is 0.5%.

In addition to the active agent(s), the cyclodextrin and the xanthan gum ingredients, the compositions of the present invention may contain one or more conventional excipients, including, but not limited to, tonicity agents, preservatives, antioxidants, chelating agents and preservative enhancing agents.

The compositions may contain an ionic or nonionic tonicity agent. The amount of tonicity agent will depend on the desired tonicity for the final formulation, but will generally be an amount sufficient to cause the formulations to have an osmolality of about 100-600 mOsm. In cases where the composition is intended for topical ophthalmic use, the composition preferably has an osmolality of about 250-350 mOsm.

The compositions of the present invention may be prepared without a preservative as a “unit-dose” or “unpreserved” formulation. If a preserved or “multi-dose” formulation is desired, the formulations may contain an ophthalmically, otically or nasally acceptable preservative, such as benzyl alcohol or quaternary ammonium halides. Quaternary ammonium halide preservatives are preferred. Suitable quaternary ammonium halide preservatives include polyquaternium-1 and benzalkonium halides. Preferred benzalkonium halides are benzalkonium chloride (“BAC”) and benzalkonium bromide. In general, the amount of the preservative ingredient will range from about 0.005-0.2. In the case where the preservative is BAC, it is preferably present at a concentration of 0.01%. In the case where the preservative is polyquaternium-1, it is preferably present at a concentration of 0.005%.

If desired, a chelating agent may also be added to the formulations of the present invention. Suitable chelating agents include edetate disodium (“EDTA”); edetate trisodium; edetate tetrasodium; and diethyleneamine pentaacetate. Most preferred is EDTA. The chelating agent, if any, will typically be present in an amount from about 0.001-0.2%. In the case of EDTA, the chelating agent is preferably present at a concentration of 0.01%.

In the case of preserved or multi-dose formulations, the solution formulations of the present invention may contain boric acid, as a component of a buffer and/or as a preservative adjunct, typically in an amount from 0.1-1.5%.

The solution formulations of the present invention are intended for topical administration to the eye, ear or nose.

The following examples are intended to illustrate, but not limit, the present invention.

### EXAMPLE 1

The formulations shown in Table 1 were prepared as follows.
1. In a suitable container dissolve in the following order the respective amount of hydroxy-propyl-β-cyclodextrin, dexamethasone, ciprofloxacin and EDTA in purified water (approx. 50% of batch weight).
2. Add the respective amount of any xanthan gum stock solution (1.2%) or polyethylene glycol until total dispersion.
3. Add the required amounts of sodium chloride and benzalkonium chloride.
4. Increase the batch weight to 90% of final batch weight.
5. Adjust pH with HCl and/or tromethamine solutions to 4.5+/−0.2
6. QS to final batch weight with purified water.
7. Autoclave formulation for 30 min (standard liquid cycle) or filter through a 0.22 μm filter.

TABLE 1

| | A | B | C | D | E | F |
|---|---|---|---|---|---|---|
| Ciproflox- | 0.35% | 0.35% | 0.35% | 0.35% | 0.35% | 0.35% |
| acin | | | | | | |
| HCl—H₂O | | | | | | |
| Dexameth- | 0.1% | 0.1% | 0.1% | 0.1% | 0.1% | 0.1% |
| asone | | | | | | |
| HPBCD | 5% | 5% | 5% | 5% | 5% | 5% |
| Edetate | 0.01% | 0.01% | 0.01% | 0.01% | 0.01% | 0.01% |
| Disodium | | | | | | |
| NaCl | — | 0.6% | 0.6% | 0.6% | 0.6% | 0.6% |
| Polyethylene | — | — | 0.25% | — | — | — |
| glycol 400 | | | | | | |
| Polyethylene | — | — | — | — | 0.25% | — |
| glycol 3350 | | | | | | |
| Polyethylene | — | — | — | — | — | 0.25% |
| glycol 8000 | | | | | | |
| Xanthan gum | — | — | — | 0.25% | — | — |
| Benzalkonium | 0.01% | 0.01% | 0.01% | 0.01% | 0.01% | 0.01% |
| Chloride | | | | | | |
| Trometh- | q.s. pH | q.s. pH | q.s. pH | q.s. pH | q.s. pH | q.s. pH |
| amine/HCl | 4.5 ± | 4.5 ± | 4.5 ± | 4.5 ± | 4.5 ± | 4.5 ± |
| | 0.2 | 0.2 | 0.2 | 0.2 | 0.2 | 0.2 |
| Purified | q.s. 100 | q.s. 100 | q.s. 100 | q.s. 100 | q.s. 100 | q.s. 100 |
| Water | | | | | | |
| Sterilization | Filter | Filter | Filter | Autoclave | Filter | Filter |

### EXAMPLE 2

Formulations A-F were evaluated to determine whether they were physically stable. Samples of each formulation (5 mL fill in clear glass scintillation vials, duplicates) were placed in a refrigerator (0° C.), pulled at the indicated time points and their physical appearance noted. The results are shown in Table 2.

TABLE 2

| Formulation | 1 day | 7 days | 14 days | 28 days |
|---|---|---|---|---|
| A | Clear, no | Clear, some | Clear, | Clear, heavy |
| | precipitate | crystals | precipitate | precipitate |
| B | Clear, no | Clear, some | Hazy, | Hazy, lots of |
| | precipitate | crystals | precipitate | crystals |
| C | Clear, no | Clear, no | Clear, some | Hazy, crystals |
| | precipitate | precipitate | crystals | suspended |
| D* | Hazy, no | Hazy, no | Hazy, no | Hazy, no |
| | precipitate | precipitate | precipitate | precipitate |
| E | Clear, no | Clear, no | Clear, few | Clear, some |
| | precipitate | precipitate | crystals | crystals |
| F | Clear, no | Clear, some | Hazy, | Hazy, lots of |
| | precipitate | crystals | precipitate | crystals |

*Initial formulation is hazy

The results in Table 2 show that in each of Formulations A-C, E and F precipitates or crystals were observed by 14 days, and in some cases after just 7 days. In contrast, no precipitates or crystals were observed in Formulation D after 28 days.

### EXAMPLE 3

A representative composition according to the invention is shown below.

| Ingredients | w/w % |
|---|---|
| Moxifloxacin HCl | 0.545 |
| Dexamethasone Alcohol | 0.1 |
| HPβCD | 5.0 |
| Xanthan Gum | 0.25 |
| NaCl | 0.3 |
| Boric Acid | 0.64 |
| Hydrochloric Acid and/or | q.s. pH to |
| Sodium Hydroxide | 5.5 ± 0.2 |
| Purified Water | q.s. to 100 |

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

What is claimed is:

1. An ophthalmic, otic or nasal composition consisting of
a) a pharmaceutically effective amount of a water-insoluble corticosteroid,
b) a cyclodextrin in an amount sufficient to solubilize the corticosteroid,
c) xanthan gum, and
d) water,
wherein said composition is a liquid and has an osmolality from about 100 to about 600 mOsm.

2. The composition of claim 1 wherein the corticosteroid is selected from the group consisting of dexamethasone; fluorometholone; prednisolone; loteprednol; and rimexolone.

3. The composition of claim 1 wherein the pharmaceutically effective amount of the water-insoluble corticosteroid is about 0.01-0.3%.

4. The composition of claim 3 wherein the pharmaceutically effective amount of the water-insoluble corticosteroid is about 0.05-0.2%.

5. The composition of claim 1 wherein the cyclodextrin is hydroxpropyl-β-cyclodextrin.

6. The composition of claim 1 wherein the amount of cyclodextrin is about 1 to 15%.

7. The composition of claim 1 wherein the osmolality is about 250 to 350 mOsm.

8. The composition of claim 1 wherein the amount of xanthan gum is about 0.1 to about 0.6%.

9. The composition of claim 8 wherein the amount of xanthan gum is about 0.1-0.4% (w/w).

10. An ophthalmic, otic or nasal composition consisting of
a) a pharmaceutically effective amount of a water-insoluble corticosteroid,
b) a cyclodextrin in an amount sufficient to solubilize the corticosteroid,
c) xanthan gum,
d) water, and
e) one or more excipients selected from the group consisting of tonicity agents; preservatives; antioxidants; chelating agents; preservative enhancing agents; buffering agents; and pH-adjusting agents,
wherein said composition is a liquid and has an osmolality from about 100 to about 600 mOsm.

11. The composition of claim 10 wherein the pharmaceutically effective amount of the water-insoluble corticosteroid is about 0.01-0.3%.

12. The composition of claim 10 wherein the cyclodextrin is hydroxypropyl-β-cyclodextrin.

13. The composition of claim 10 wherein the amount of cyclodextrin is about 1 to 15%.

14. The composition of claim 10 wherein the amount of xanthan gum is about 0.1 to about 0.6%.

15. An ophthalmic, otic or nasal composition consisting of
a) a pharmaceutically effective amount of a water-insoluble corticosteroid,
b) a cyclodextrin in an amount sufficient to solubilize the corticosteroid,
c) xanthan gum,
d) water,
e) one or more excipients selected from the group consisting of tonicity agents; preservatives; antioxidants; chelating agents; preservative enhancing agents; buffering agents; and pH-adjusting agents, and
f) a second active ingredient suitable for ophthalmic, otic or nasal administration,
wherein said composition is a liquid and has an osmolality from about 100 to about 600 mOsm.

16. The composition of claim 15 wherein the pharmaceutically effective amount of the water-insoluble corticosteroid is about 0.01-0.3%.

17. The composition of claim 15 wherein the cyclodextrin is hydroxypropyl-β-cyclodextrin.

18. The composition of claim 15 wherein the amount of cyclodextrin is about 1 to 15%.

19. The composition of claim 15 wherein the amount of xanthan gum is about 0.1 to about 0.6%.

20. The composition of claim 15 wherein the second active ingredient is a fluoroquinolone antibiotic drug.
